(19) United States
(12) Patent Application Publication
(10) Pub. No.: US 2009/0149634 A1
(43) Pub. Date: Jun. 11, 2009

(54) POROUS BODY COMPRISING APATITE/COLLAGEN COMPOSITE FIBERS AND ITS PRODUCTION METHOD

(75) Inventors: Daisuke SHOJI, Tokyo (JP); Naomi MOCHIZUKI, Saitama (JP); Tomoji TAKAYAMA, Tokyo (JP); Yuko KOZAKA, Saitama (JP); Kenichi SHINOMIYA, Tokyo (JP); Shinichi SOTOME, Tokyo (JP); Yuichi KAWASAKI, Tokyo (JP)

Correspondence Address: GREENBLUM & BERNSTEIN, P.L.C., 1950 ROLAND CLARKE PLACE, RESTON, VA, 20191, US

(73) Assignees: HOYA CORPORATION, Tokyo (JP); TOKYO MEDICAL AND DENTAL UNIVERSITY, Tokyo (JP)

(21) Appl. No.: 12/260,275

(22) Filed: Oct. 29, 2008

(30) Foreign Application Priority Data
Nov. 1, 2007 (JP) 2007-285469

(51) Int. Cl. C07K 14/78

(52) U.S. Cl. 530/356

(57) ABSTRACT

A porous body comprising apatite/collagen composite fibers, which has a half-value period of strength of 0.8-1.6 hours, the half-value period of strength being the time until the strength of the porous body of 10 mm×10 mm×4 mm comprising apatite/collagen composite fibers is reduced to half, after the porous body degassed by pressure reduction to 3 kPa (absolute pressure) for 10 minutes in a phosphate buffer saline is given a 20-% strain at a speed of 10 mm/minute.

### FIELD OF THE INVENTION

The present invention relates to a porous body comprising apatite/collagen composite fibers having an optimum half-value period of strength for bone formation and suitable for artificial bone, cell scaffolds, etc., and its production method.

### BACKGROUND OF THE INVENTION

Because artificial bone made of apatite having compatibility with human bone can be bonded to the human bone directly, it has recently been finding clinical applications in cosmetic surgery, neurosurgery, plastic surgery, oral surgery, etc. Because mechanical strength and biocompatibility are substantially in an inversely proportional relation in a porous body composed of apatite and collagen, the larger the mechanical strength, the smaller the biocompatibility. Because the mechanical strength and biocompatibility of the porous body comprising apatite/collagen composite fibers can be controlled by composition, porosity, pore size, etc. to some extent, their optimum balance can be designed depending on applications. However, because there are recently many applications of artificial bone, it has become difficult to obtain porous bodies satisfactory for all applications only by controlling their compositions, porosities, pore sizes, etc.

JP 11-513590 A discloses a porous matrix decomposable in the human body comprising insoluble biopolymer fibers, a binder and calcium phosphate, which keeps its physical shape for at least about 3 days after implanted in a biological environment in which bone substitution occurs, and also keeps its porosity for about 7-14 days. However, because this porous matrix has low mechanical strength, it cannot be handled easily at an operation site. In addition, it is absorbed too quickly after the operation, resulting in too quick reduction of biocompatibility.

### OBJECT OF THE INVENTION

Accordingly, an object of the present invention is to provide a porous body comprising apatite/collagen composite fibers having an optimum balance of mechanical strength and biocompatibility and suitable for artificial bone, cell scaffolds, etc., and its production method.

### DISCLOSURE OF THE INVENTION

As a result of extensive investigation in view of the above object, the inventors have found that a porous body comprising apatite/collagen composite fibers having excellent balance of mechanical strength and biocompatibility in the human body can be obtained by regulating its half-value period of strength in a predetermined range. The present invention has been completed based on such findings.

Thus, the porous body comprising apatite/collagen composite fibers according to the present invention has a half-value period of strength of 0.8-1.6 hours, the half-value period of strength being the time until the strength of the porous body of 10 mm×10 mm×4 mm comprising apatite/collagen composite fibers is reduced to half, after the porous body degassed by pressure reduction to 3 kPa (absolute pressure) for 10 minutes in a phosphate buffer saline is given a 20-% strain at a speed of 10 mm/minute.

The half-value period of strength is preferably 0.9-1.5 hours. The mass ratio of the apatite to the collagen is preferably 9/1-6/4.

The porous body is preferably irradiated with γ rays in a dose of 10-42 kGy. The irradiation dose of γ rays is more preferably 16-35 kGy.

The method of the present invention for producing a porous body comprising apatite/collagen composite fibers, which has a half-value period of strength of 0.8-1.6 hours, the half-value period of strength being the time until the strength of the porous body of 10 mm×10 mm×4 mm comprising apatite/collagen composite fibers is reduced to half, after the porous body degassed by pressure reduction to 3 kPa (absolute pressure) for 10 minutes in a phosphate buffer saline is given a 20-% strain at a speed of 10 mm/minute, comprises the steps of obtaining the porous body comprising apatite/collagen composite fibers by freeze-drying, and irradiating the porous body with γ rays in a dose of 10-42 kGy.

A cross-linking treatment is preferably conducted before the irradiation of γ rays.

### BRIEF DESCRIPTION OF THE DRAWINGS

FIG. 1 is a schematic view showing a texture analyzer for measuring the strength change of a porous body comprising apatite/collagen composite fibers.

FIG. 2 is a graph showing the strength change of a porous body comprising apatite/collagen composite fibers after a 20-% strain is given.

FIG. 3 is a graph showing the relation between the irradiation dose of γ-rays and the half-value period of strength in Samples 1-3.

FIG. 4 is a graph showing the strength change of the porous bodies of Samples 1-4 comprising apatite/collagen composite fibers after a 20-% strain is given.

FIG. 5(a) is an optical photomicrograph showing the porous body comprising apatite/collagen composite fibers of Sample 5 (irradiation of γ rays: 0 kGy), which was stained with HE two weeks after embedded in a rat bone.

FIG. 5(b) is an optical photomicrograph showing the porous body comprising apatite/collagen composite fibers of Sample 7 (irradiation of γ rays: 16 kGy), which was stained with HE two weeks after embedded in a rat bone.

FIG. 5(c) is an optical photomicrograph showing the porous body comprising apatite/collagen composite fibers of Sample 8 (irradiation of γ rays: 25 kGy), which was stained with HE two weeks after embedded in a rat bone.

FIG. 5(d) is an optical photomicrograph showing the porous body comprising apatite/collagen composite fibers of Sample 9 (irradiation of γ rays: 35 kGy), which was stained with HE two weeks after embedded in a rat bone.

FIG. 5(e) is an optical photomicrograph showing the porous body comprising apatite/collagen composite fibers of Sample 10 (irradiation of γ rays: 50 kGy), which was stained with HE two weeks after embedded in a rat bone.

FIG. 6(a) is an optical photomicrograph showing the porous body comprising apatite/collagen composite fibers of Sample 5 (irradiation of γ rays: 0 kGy) and osteoclasts, which were stained with TRAP two weeks after embedded in a rat bone, and its schematic view.

FIG. 6(b) is an optical photomicrograph showing the porous body comprising apatite/collagen composite fibers of Sample 7 (irradiation of γ rays: 16 kGy) and osteoclasts, which were stained with TRAP two weeks after embedded in a rat bone, and its schematic view.

FIG. 6(c) is an optical photomicrograph showing the porous body comprising apatite/collagen composite fibers of Sample 8 (irradiation of γ rays: 25 kGy) and osteoclasts, which were stained with TRAP two weeks after embedded in a rat bone, and its schematic view.

FIG. 6(d) is an optical photomicrograph showing the porous body comprising apatite/collagen composite fibers of Sample 9 (irradiation of γ rays: 35 kGy) and osteoclasts, which were stained with TRAP two weeks after embedded in a rat bone, and its schematic view.

FIG. 6(e) is an optical photomicrograph showing the porous body comprising apatite/collagen composite fibers of Sample 10 (irradiation of γ rays: 50 kGy) and osteoclasts, which were stained with TRAP two weeks after embedded in a rat bone, and its schematic view.

FIG. 7(a) is an optical photomicrograph showing the porous body comprising apatite/collagen composite fibers of Sample 5 (irradiation of γ rays: 0 kGy) two weeks after embedded in a muscle layer.

FIG. 7(b) is an optical photomicrograph showing the porous body comprising apatite/collagen composite fibers of Sample 7 (irradiation of γ rays: 16 kGy) two weeks after embedded in a muscle layer.

FIG. 7(c) is an optical photomicrograph showing the porous body comprising apatite/collagen composite fibers of Sample 8 (irradiation of γ rays: 25 kGy) two weeks after embedded in a muscle layer.

FIG. 7(d) is an optical photomicrograph showing the porous body comprising apatite/collagen composite fibers of Sample 9 (irradiation of γ rays: 35 kGy) two weeks after embedded in a muscle layer.

FIG. 7(e) is an optical photomicrograph showing the porous body comprising apatite/collagen composite fibers of Sample 10 (irradiation of γ rays: 50 kGy) two weeks after embedded in a muscle layer.

FIG. 8(a) is a CT photograph showing the porous body comprising apatite/collagen composite fibers of Sample 5 (irradiation of γ rays: 0 kGy) two weeks after embedded in a muscle layer.

FIG. 8(b) is a CT photograph showing the porous body comprising apatite/collagen composite fibers of Sample 7 (irradiation of γ rays: 16 kGy) two weeks after embedded in a muscle layer.

FIG. 8(c) is a CT photograph showing the porous body comprising apatite/collagen composite fibers of Sample 8 (irradiation of γ rays: 25 kGy) two weeks after embedded in a muscle layer.

FIG. 8(d) is a CT photograph showing the porous body comprising apatite/collagen composite fibers of Sample 9 (irradiation of γ rays: 35 kGy) two weeks after embedded in a muscle layer.

FIG. 8(e) is a CT photograph showing the porous body comprising apatite/collagen composite fibers of Sample 10 (irradiation of γ rays: 50 kGy) two weeks after embedded in a muscle layer.

### DESCRIPTION OF THE BEST MODE OF THE INVENTION

### [1] Half-Value Period of Strength of Porous Body

The mechanical strength of a porous body comprising apatite/collagen composite fibers in the human body depends on an apatite/collagen mass ratio, porosity, pore size, etc., but investigation has revealed that it also depends on the conditions of post-treatments such as γ rays irradiation, etc. It has further been found that porous body having a half-value period of strength of 0.8-1.6 hours has mechanical strength and biocompatibility in an optimum balance for bone formation. Namely, the porous body comprising apatite/collagen composite fibers, which has optimum mechanical strength and biocompatibility for bone formation, can be designed by measuring the half-value period of strength, and the half-value period of strength can be controlled by the adjustment of the irradiation dose of γ-rays. As a result, even porous bodies obtained under the same production conditions can be provided with mechanical strength and biocompatibility adjusted for applications. The preferred half-value period of strength is 0.9-1.5 hours.

The half-value period of strength of the porous body is measured by the following method.

(1) Removal of Air from Porous Body

The porous body comprising apatite/collagen composite fibers (10 mm×10 mm×4 mm) is immersed in a phosphate buffer saline (PBS), subjected to pressure reduction to 3 kPa (absolute pressure) for 10 minutes to remove air from the porous body, and then returned to the atmospheric pressure.

(2) Application of Initial Strain

As shown in FIG. 1, the pressure P is applied to the air-removed porous body 1 immersed in PBS 3 at a speed of 10 mm/minute with a sample-pushing part 4 of 20 mm in diameter in a texture analyzer, until the porous body 1 undergoes a strain of 20%. The compression strength of the porous body 1 at a strain of 20% is measured as an initial strength by a detector (not shown).

(3) Measurement of Change of Strength with Time

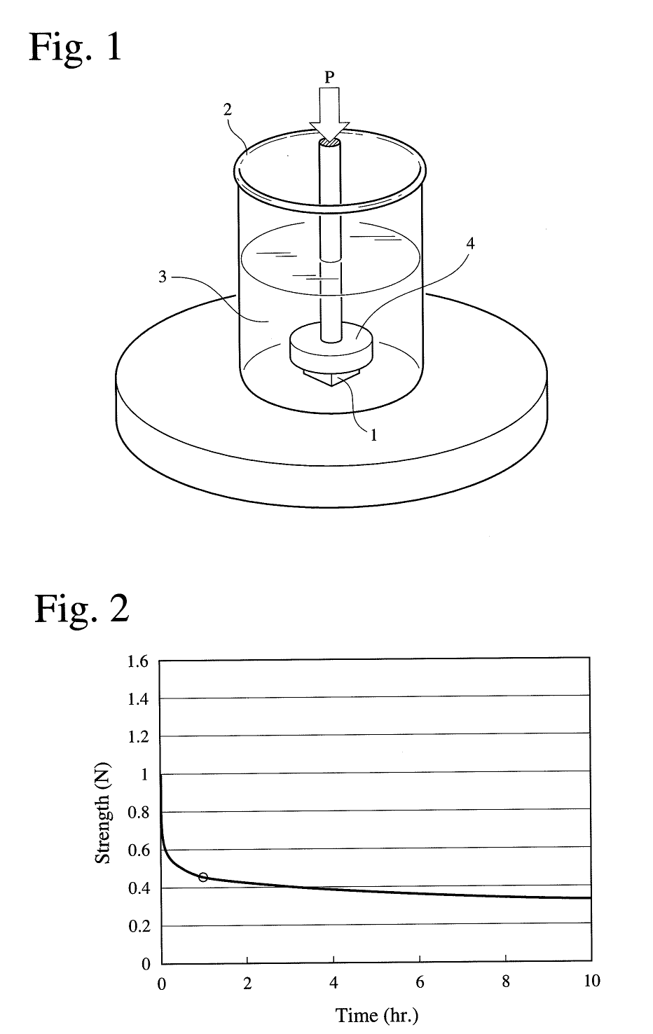

After removing the pressure P, the compression strength (corresponding to strain) of the porous body 1 is measured for 10 hours to determine a time when the compression strength is reduced to half of the initial strength (half-value period of strength). FIG. 2 shows one example of the change of compression strength with time, with the half-value period indicated by a circle.

When the porous body has too short a half-value period of strength, the porous body embedded in the human body is absorbed before a new bone is formed. On the other hand, when the half-value period of strength is too long, the porous body remains unabsorbed, hindering the formation of a new bone. Because the porous body of the present invention has an optimum half-value period of strength, the absorption of the porous body into the human body and the formation of a new bone occur in an optimum balance.

### [2] Porous Body Comprising Apatite/Collagen Composite Fibers

The porous body of the present invention comprising apatite/collagen composite fibers is composed of pluralities of fiber layers each comprising apatite/collagen composite fibers. The fiber layers are in a plate shape of about 10-500 μm in thickness, and overlapping randomly. There are pillars constituted by apatite/collagen composite fibers between the fiber layers. Because the fiber layers are microscopically supported only by dispersed pillars in a lamination direction, it is considered that the mechanical strength of the porous body comprising apatite/collagen composite fibers is low in a lamination direction but high in a layer direction. However, because the fiber layers are overlapping randomly as described above, the overlapping directions of the fiber layers are averaged macroscopically, resulting in substantially no anisotropy of strength.

Plate-like pores are defined by the fiber layers and the pillars dispersed therebetween. The pores are as thick as about 0.5-10 times the fiber layers. When this porous body comprising apatite/collagen composite fibers is embedded in the human body, blood vessels, relatively large proteins, etc. easily enter the substantially plate-like pores, accelerating the formation of bone.

### [3] Production Method of Porous Body Comprising Apatite/Collagen Composite Fibers

(1) Apatite/Collagen Composite Fibers

(a) Starting Materials

Starting materials for the apatite/collagen composite fibers are collagen, phosphonic acid or its salts, and calcium salts. Though not particularly restricted, the collagen may be extracted from animals, etc. The kinds, parts, ages, etc. of the animals are not particularly restrictive. In general, collagen obtained from skins, bones, cartilages, tendons, internal organs, etc. of mammals such as cow, pig, horse, rabbit and rat, and birds such as hen, etc. may be used. Collagen-like proteins obtained from skins, bones, cartilages, fins, scales, internal organs, etc. of fish such as cod, flounder, flatfish, salmon, trout, tuna, mackerel, red snapper, sardine, shark, etc. may also be used. The extraction method of collagen is not particularly restrictive but may be a usual one.

Phosphoric acid or its salts [hereinafter referred to simply as “phosphoric acid (salt)”] include phosphoric acid, disodium hydrogenphosphate, sodium dihydrogenphosphate, dipotassium hydrogenphosphate, and potassium dihydrogenphosphate. The calcium salts include calcium carbonate, calcium acetate, and calcium hydroxide. The phosphate and the calcium salt are preferably added in the form of a uniform aqueous solution or suspension.

A mass ratio of the apatite-forming materials [phosphoric acid (salt) and calcium salts] to the collagen may be determined properly depending on the target composition of the apatite/collagen composite fibers. The apatite/collagen mass ratio is preferably 9/1-6/4, particularly about 8/2. With the apatite/collagen mass ratio of more than 9/1 or less than 6/4, it is likely difficult to provide the porous body with a half-value period of strength within the above range, not suitable for scaffolds.

(b) Preparation of Solution

The concentrations of an aqueous phosphoric acid (salt) solution and an aqueous calcium salt solution are not particularly restricted as long as the phosphoric acid (salt) and the calcium salt are in desired proportions, but it is preferable for the convenience of a dropping operation described below that the concentration of the aqueous phosphoric acid (salt) solution is about 50-250 mM, and that the concentration of the aqueous calcium salt solution is about 200-600 mM. The collagen generally in the form of an aqueous phosphoric acid solution is added to the aqueous phosphoric acid (salt) solution. In the aqueous solution of collagen in phosphoric acid, the concentration of the collagen is preferably 0.5-1% by mass, more preferably 0.8-0.9% by mass, particularly about 0.85% by mass, while the concentration of the phosphoric acid is preferably 10-30 mM, more preferably 15-25 mM, particularly about 20 mM.

(c) Production of Apatite/Collagen Composite Fibers

Water in an amount of preferably 0.5-2 times, more preferably 0.8-1.2 times, particularly in substantially the same amount as that of the aqueous calcium salt solution, is introduced into a reaction vessel, and heated to about 40° C., and an aqueous phosphoric acid (salt) solution containing collagen and an aqueous calcium salt solution are simultaneously dropped into the water. The length of apatite/collagen composite fibers can be controlled by the dropping conditions. The dropping speed is preferably about 10-50 ml/minute, and the reaction solution is preferably stirred at about 50-300 rpm. During the dropping, it is preferable to keep the concentrations of calcium ions and phosphoric acid ions in the reaction solution to 3.75 mM or less and 2.25 mM or less, respectively, to keep the reaction solution at pH of 8.9-9.1. When the concentrations of calcium ions and/or phosphoric acid ions are too high, the porous body is not self-organized. The term “self-organization” used herein means that hydroxyapatite (calcium phosphate having an apatite structure) has orientation peculiar to living bone along collagen fibers, namely that the C-axis of the hydroxyapatite is in alignment with the collagen fibers. Under the above dropping conditions, the apatite/collagen composite fibers are self-organized to a length of 1 mm or less suitable for the porous body.

After the completion of dropping, a slurry-like dispersion containing the apatite/collagen composite fibers is freeze-dried. The freeze-drying can be carried out by rapid drying in vacuum in a frozen state at −10° C. or lower.

(2) Preparation of Dispersion Containing Apatite/Collagen Composite Fibers

The apatite/collagen composite fibers are mixed with water, an aqueous phosphoric acid solution, etc. and stirred to prepare a paste-like dispersion. The amount of a liquid contained in this dispersion is preferably 80-99% by volume, more preferably 90-97% by volume. Namely, the amount of the composite fibers is preferably 1-20% by volume, more preferably 3-10% by volume. Steam is preferably attached to the apatite/collagen composite fibers in advance. In this case, the amount of water to be added should be determined with the amount of steam attached to the apatite/collagen composite fibers subtracted.

The resultant porous body has porosity P (%), which depends on a volume ratio of the apatite/collagen composite fibers to the liquid in the dispersion as represented by the following formula (1):

P=Y/(X+Y)×100 (1),

wherein X represents the volume of the apatite/collagen composite fibers in the dispersion, and Y represents the volume of the liquid in the dispersion. Accordingly, it is possible to control the porosity P of the porous body by adjusting the amount of the liquid to be added. The apatite/collagen composite fibers are cut to have a wide length distribution by stirring the dispersion after adding the liquid, resulting in a porous body with improved strength.

Collagen as a binder is added to the dispersion of apatite/collagen composite fibers, and further stirred. The amount of the collagen added is preferably 1-10% by mass, more preferably 3-6% by mass, based on 100% by mass of the composite fibers. As in the case of the composite fibers, the collagen is added preferably in the form of an aqueous phosphoric acid solution. Though the concentration of the aqueous solution of collagen in phosphoric acid is not particularly restricted, it is practical that the concentration of the collagen is 0.8-0.9% by mass (for instance, 0.85% by mass), and that the concentration of phosphoric acid is 15-25 mM (for instance, 20 mM).

(3) Gelation of Dispersion

The dispersion turned acidic by the addition of collagen dissolved in an aqueous phosphoric acid (salt) solution is mixed with a sodium hydroxide solution to adjust its pH to preferably 6.8-7.6, more preferably 7.0-7.4, particularly about 7. By adjusting the pH of the dispersion to 6.8-7.6, the collagen added as a binder is quickly turned to fibers.

The phosphate buffer solution (PBS) as concentrated as about 2.5-10 times is added to the dispersion and stirred to adjust its ion strength to about 0.2-0.8. The larger ion strength of the dispersion accelerates the collagen added as a binder to be turned to fibers.

The dispersion charged into a molding die is kept at a temperature of 35° C. to 43° C. for gelation. For sufficient gelation of the dispersion, the heating time is preferably 0.5 to 3.5 hours, more preferably 1 to 3 hours. With the dispersion kept at a temperature of 35-43° C., the collagen added as a binder is turned to fibers, resulting in the gelation of the dispersion. The gelled dispersion can prevent the apatite/collagen composite fibers from precipitating therein, thereby producing a uniform porous body.

(4) Freezing and Drying of Gel

The gel containing the apatite/collagen composite fibers is frozen. The average pore diameter of a porous body comprising apatite/collagen composite fibers to be obtained depends on the gel-freezing time. The freezing temperature is preferably −100° C. to 0° C., more preferably −100° C. to −10° C., particularly −80° C. to −20° C. When it is lower than −100° C., the resultant porous body comprising apatite/collagen composite fibers has too small an average pore diameter.

The solidified gel is freeze-dried to a porous body. The freeze-drying is conducted by evacuating the frozen gel at −10° C. or lower, and rapidly drying it, as in the case of the apatite/collagen composite fibers. The freeze-drying need only be conducted until the dispersion is fully dried, so the freezing time is not particularly restricted, but it is generally about 24-72 hours.

(5) Cross-Linking of Collagen

The cross-linking of collagen may be carried out by any methods such as physical cross-linking methods using γ-rays, ultraviolet rays, thermal dehydration, electron beams, etc., or chemical cross-linking methods using cross-linking agents, condensation agents, etc. In the case of the chemical cross-linking, the porous body is immersed in a cross-linking agent solution. The cross-linking agents may be, for instance, aldehydes such as glutaraldehyde, formaldehyde, etc.; isocyanates such as hexamethylene diisocyanate, etc.; carbodiimides such as a hydrochloric acid salt of 1-ethyl-3-(3-dimethylaminopropyl)carbodiimide; polyepoxides such as ethylene glycol diethyl ether, etc.; transglutaminase, etc. Among these cross-linking agents, glutaraldehyde is preferable from the aspects of the easiness of controlling the degree of cross-linking and the biocompatibility of the resultant porous body.

When cross-linking is conducted by using glutaraldehyde, the concentration of a glutaraldehyde solution is preferably 0.005 to 0.015% by mass, more preferably 0.005 to 0.01% by mass. When alcohol such as ethanol, etc. is used as a solvent for the glutaraldehyde solution, the cross-linking of collagen and the dehydration of the porous body may be conducted simultaneously. When the dehydration and the cross-linking are conducted simultaneously, the cross-linking reaction of collagen occurs in a state where the apatite/collagen composite fibers are contracted, resulting in a porous body with improved elasticity.

After the cross-linking, the porous body is immersed in an aqueous solution of about 2% by mass of glycine to remove unreacted glutaraldehyde, and then washed with water. The porous body is further immersed in ethanol for dehydration, and then dried at room temperature.

In the case of cross-linking by thermal dehydration, the freeze-dried porous body may be kept at 100° C. to 160° C. and 0-100 hPa for 10-12 hours in a vacuum oven.

### [3] Irradiation of γ Rays

The cross-linked porous body is preferably irradiated with γ rays in a dose of 10-42 kGy. A γ-ray source is preferably cobalt 60. With the irradiation dose of γ-rays less than 10 kGy or more than 42 kGy, it is likely difficult to provide the porous body with a half-value period of strength within the above range, not suitable for scaffolds. The irradiation may be conducted by an incremental irradiation method of repeating the steps of introducing the porous body into an irradiation chamber by a belt conveyor, taking it out of the chamber after a predetermined period of time, and then introducing it into the chamber again until reaching a predetermined amount of irradiation, a stationary method of conducting the irradiation of γ rays to the porous body placed in an irradiation chamber, etc.

The present invention will be described in detail with reference to Examples below without intension of limitation.

### EXAMPLE 1

### Production of Samples 1-3

(A) Synthesis of Apatite/Collagen Composite Fibers

235 g of an aqueous solution of collagen in phosphoric acid (collagen concentration: 0.85% by mass, phosphoric acid concentration: 20 mM) was added to 168 ml of a 120-mM aqueous phosphoric acid solution, and stirred to prepare an aqueous solution of collagen in phosphoric acid. Also, 200 ml of a 400-mM calcium hydroxide suspension was prepared. The aqueous solution of collagen in phosphoric acid and the calcium hydroxide suspension were simultaneously dropped both at a speed of about 30 ml/minute into a 200 ml of purified water heated at 40° C. in a reaction vessel, and stirred at 200 rpm to prepare slurry containing apatite/collagen composite fibers. During dropping, the pH of a reaction liquid was kept at 8.9-9.1. The resultant apatite/collagen composite fibers had length of substantially 1 mm or less and an apatite/collagen mass ratio of 8/2.

(B) Production of Cross-Linked, Porous Apatite/Collagen Body

3.6 ml of purified water was added to 1 g of apatite/collagen composite fibers obtained by freeze-drying the above slurry, and stirred to prepare a paste-like dispersion. After 4 g of an aqueous solution of collagen in phosphoric acid was added to this paste-like dispersion and stirred, a 1-N aqueous NaOH solution was added until the pH became substantially 7. The mass ratio of the apatite/collagen composite fibers to the collagen was 97/3. PBS concentrated to 10 times was then added until the ion strength of the dispersion became 0.8. The amount of the liquid (purified water+aqueous solution of collagen in phosphoric acid+aqueous NaOH solution+PBS) was 95% by volume of the apatite/collagen composite fibers.

The resultant dispersion was charged into a molding die to carry out gelation at 37° C. for 2 hours to produce a jelly-like molding. This molding was frozen at −20° C., dried by a freeze drier, and then cross-linked by thermal dehydration at 140° C. to obtain a cross-linked, porous apatite/collagen body. A square-columnar test piece of 5 mm×5 mm×10 mm was cut out of this cross-linked, porous apatite/collagen body to measure its fracture strength at a drawing speed of 0.1 mm/second. As a result, the fracture strength of the cross-linked, porous apatite/collagen body was about 0.8 N.

(C) Irradiation of γ Rays

9 plates of 10 mm×10 mm×4 mm were cut out of the cross-linked, porous apatite/collagen body, and every three plates were irradiated with γ rays in doses of 10 kGy, 25 kGy and 50 kGy, respectively, to prepare Samples 1-3.

### Production of Sample 4

3 pieces of 10 mm×10 mm×4 mm as Sample 4 were cut out of a porous hydroxyapatite/collagen body “HEALOS” (apatite: about 20% by mass, and collagen: about 80% by mass) available from DePuy Spine, Inc.

Strength Test

The half-value periods of strength of Samples 1-4 were measured by the following method.

(1) Each Sample was immersed in a phosphate buffer saline (PBS), evacuated to 3 kPa (absolute pressure) for 10 minutes, and then returned to the atmospheric pressure.

(2) Using a texture analyzer available from Shimadzu Corporation, each Sample immersed in PBS was given a 20-% strain at a speed of 10 mm/minute (see FIG. 1). A sample-pushing part 4 had a diameter of 20 mm. The initial strength of Samples when a 20-% strain was added was 0.8-1.3 N (Sample 1), 0.9-1.7 N (Sample 2), 0.5-0.7 N (Sample 3), and 0.07-0.1 N (Sample 4).

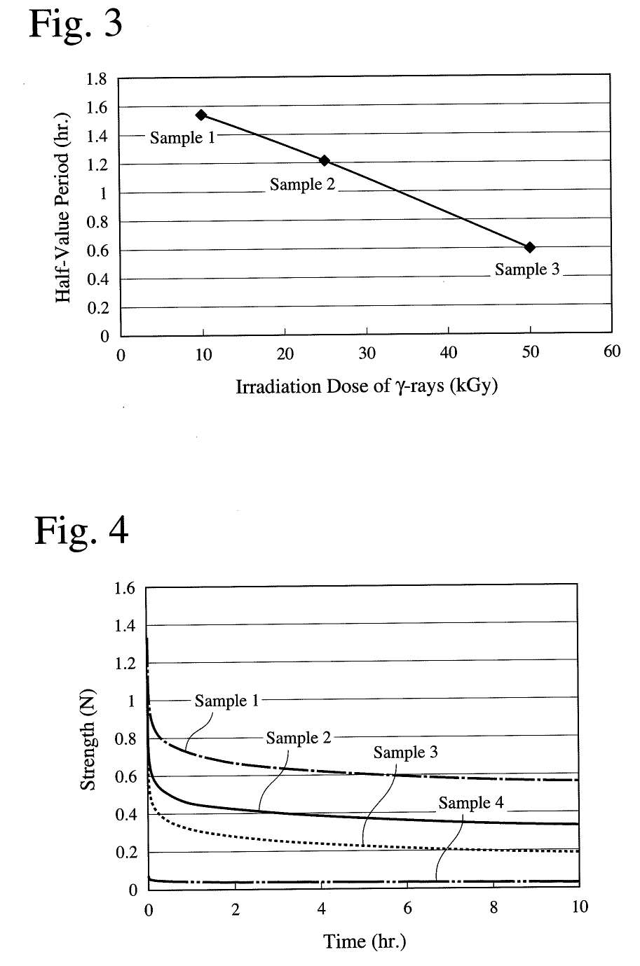

(3) The change of strength with time was measured for 10 hours after a 20-% strain was added, to determine a half-value period of strength. The measurement results are shown in Table 1 and FIGS. 3 and 4.

TABLE 1

| | |
|---|---|
| Irradiation | Half-Value Period of Strength |

| | Dose (kGy) of | | Standard |
|---|---|---|---|
| No.⁽¹⁾ | γ Rays | Hours | Deviation |
| Sample 1 | 10 | 1.54 | 0.55 |
| Sample 2 | 25 | 1.21 | 0.06 |
| Sample 3 | 50 | 0.60 | 0.11 |
| Sample 4 | No | 6 and 10 or more⁽²⁾ | — |

Note:
⁽¹⁾Samples 1 and 2 are within the range of the present invention, while Samples 3 and 4 are outside the range of the present invention.
⁽²⁾Among the three Samples, one Sample had a half-value period of 6 hours, while the other two had half-value periods of 10 hours or more.

As is clear from FIGS. 3 and 4, as the irradiation dose of γ-rays increased, the half-value period of strength shortened. Specifically, the half-value periods of strength of Samples 1 and 2 were 1.54 hours and 1.21 hours, respectively, within the range of the present invention, while the half-value period of strength of Sample 3 was about half of that of Sample 2. Sample 4 had not only extremely small initial strength, but also too long a half-value period of strength of 6-10 hours. Why Sample 4 had a long half-value period of strength (keeping elasticity for a long period of time) is due to the fact that a high percentage of collagen was contained in the porous body. Accordingly, it is likely that the collagen is prematurely absorbed after the operation, resulting in the decomposition of the porous body before the formation of bone. It has been found that to obtain the half-value period of strength within the range of the present invention, it is important to adjust not only the composition of the porous body but also the irradiation dose of γ-rays.

### EXAMPLE 2

Samples 5-10 of 2 mm×2 mm×3 mm each in the number of five were produced in the same manner as in Example 1, except that the irradiation dose of γ-rays to the porous body comprising apatite/collagen composite fibers was 0 kGy, 10 kGy, 16 kGy, 25 kGy, 35 kGy, and 50 kGy, respectively. Incidentally, Samples 6, 8 and 10 correspond to Samples 1-3 in Example 1.

(1) Measurement of Half-Value Period of Strength

The half-value periods of strength of Samples 5, 7 and 9 were measured by the same method as in Example 1. The results are shown in Table 2 together with the data of Samples 6, 8 and 10.

(2) Evaluation of Bone Formation

Each Sample 5-10 was embedded in a rat bone, taken out after two weeks, and subjected to the stain of part of the bone tissue and the chondrocytes with hematoxylin-eosin (HE), and the stain of the osteoclasts with tartrate-resistant acid phosphatase (TRAP) to evaluate bone formation in each Sample.

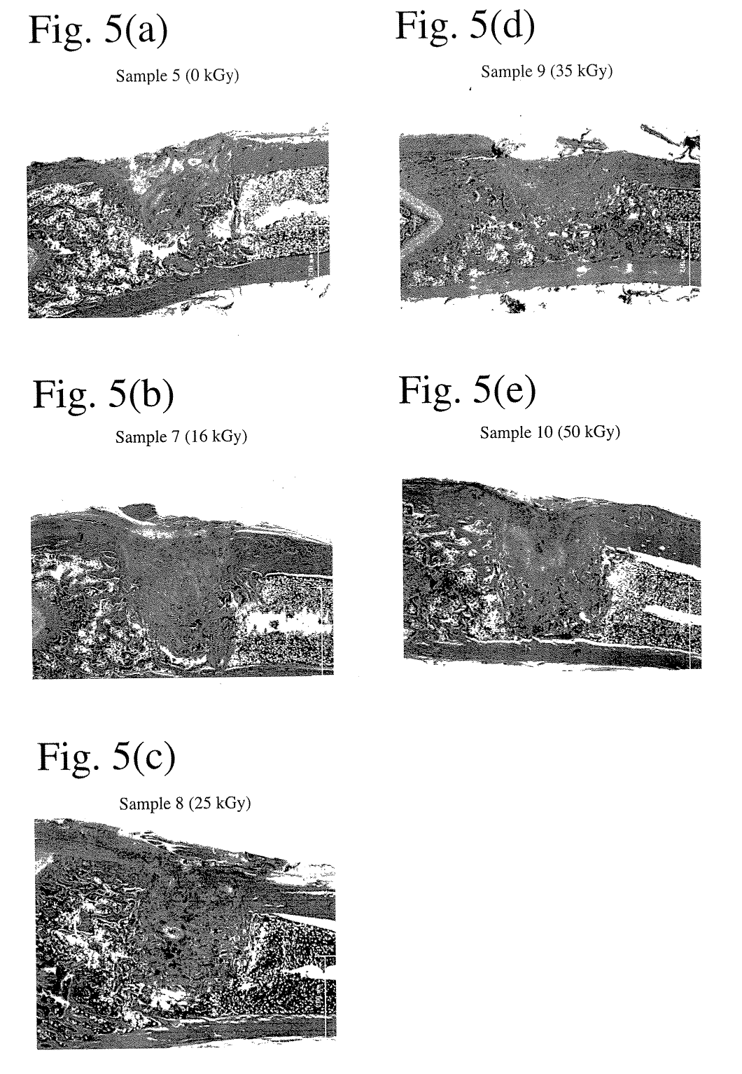

The results of the HE stain are shown in FIGS. 5(a) to 5(e). Sample 5 in which the irradiation dose of γ-rays was 0 kGy was mostly unabsorbed, substantially keeping its original shape [FIG. 5(a)]. Sample 10 in which the irradiation dose of γ-rays was 50 kGy was decomposed, resulting in a dent in a filled portion [FIG. 5(e)]. On the other hand, Samples 7-9 in which the irradiation doses of γ-rays were 16 kGy, 25 kGy and 35 kGy, respectively, were well absorbed and replaced by the bone, indicating good bone regeneration [FIGS. 5(b), 5(c) and 5(d)].

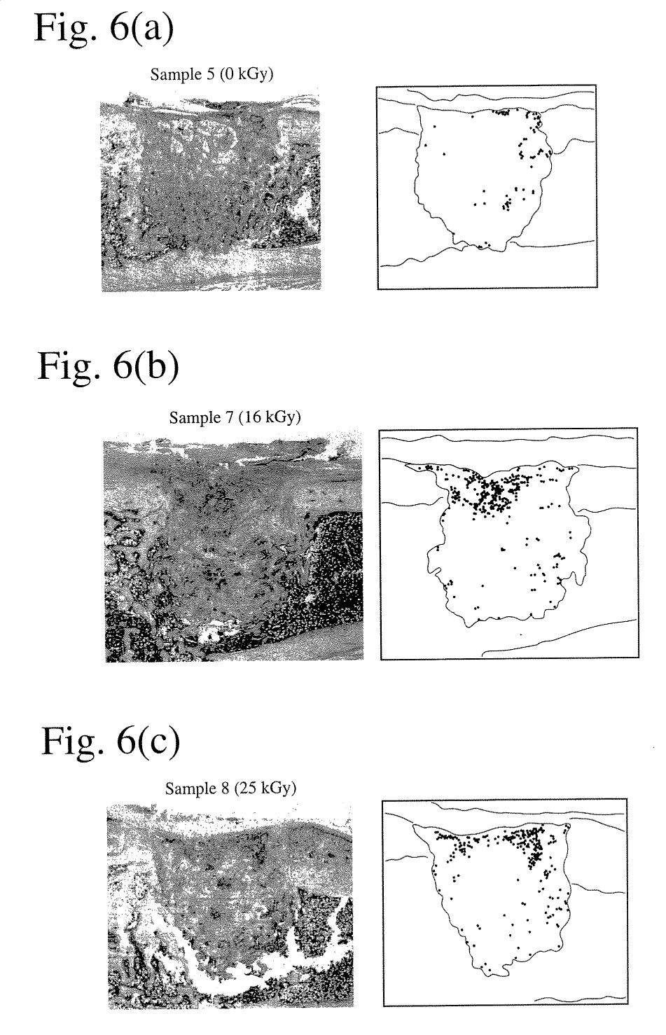

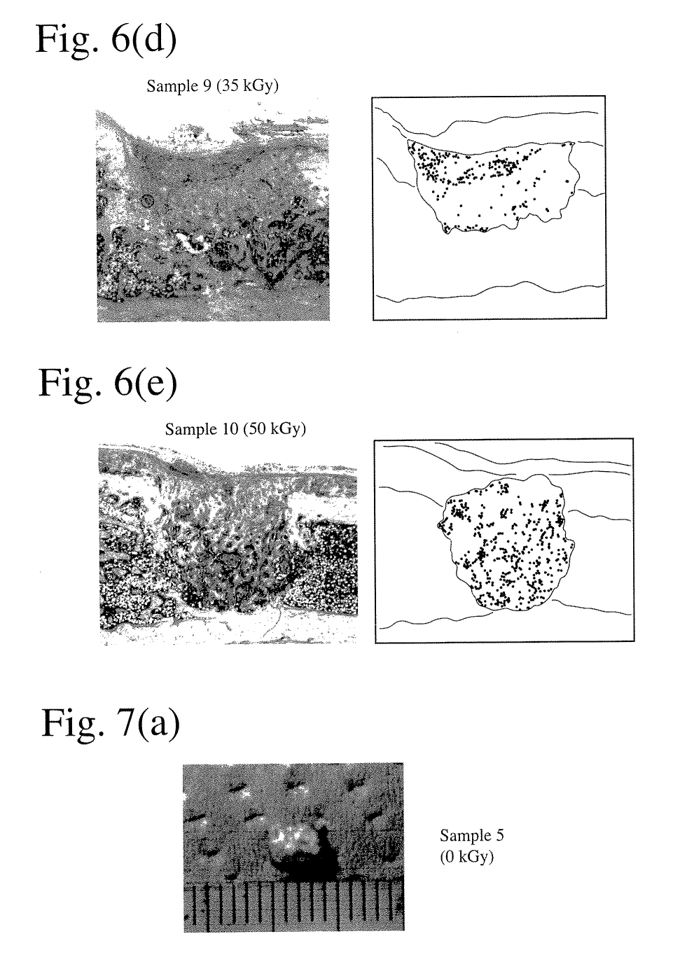

The results of the TRAP stain are shown in FIGS. 6(a) to 6(e). In the schematic view on the right side of each FIG. 6(a) to 6(e), the osteoclasts are shown by black dots. In Sample 10 in which the irradiation dose of γ-rays was 50 kGy, many osteoclasts were observed [FIG. 6(e)]. It is presumed that the osteoclasts acted excessively.

(3) Measurement of Volume Reduction Ratio

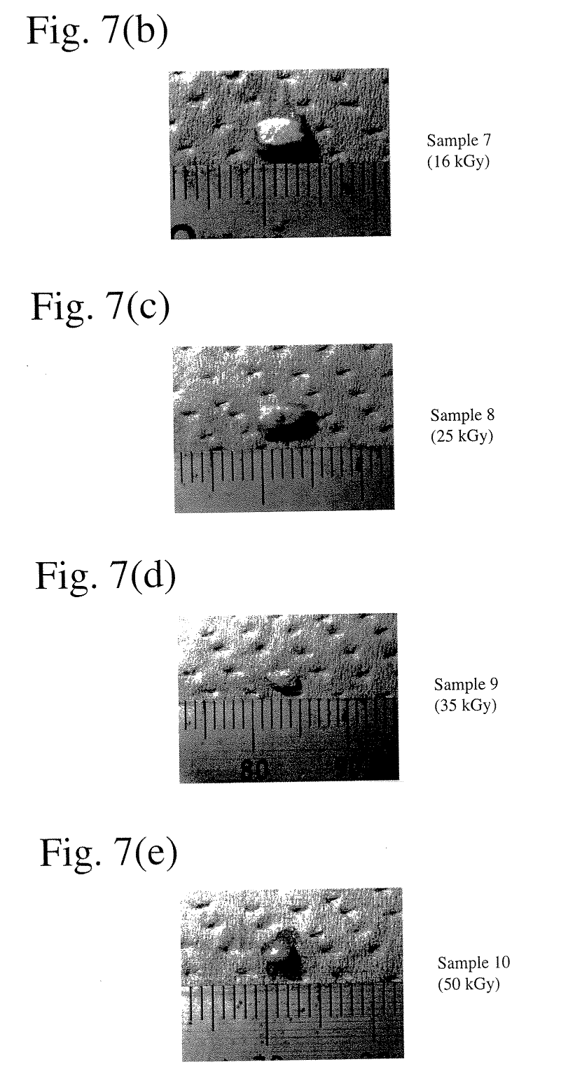

The volume reduction ratios were determined from photographs [FIGS. 7(a)-7(e)] of Samples 5-10 each embedded in a muscle layer of a rat and taken out after two weeks. The volume reduction ratios are shown in Table 2. While the volume reduction ratio of Sample 5 in which the irradiation dose of γ-rays was 0 kGy was 4% [FIG. 7(a)], the volume reduction ratio of Sample 10 in which the irradiation dose of β-rays was 50 kGy was 64% [FIG. 7(e)]. These results coincided with the evaluation results of bone formation.

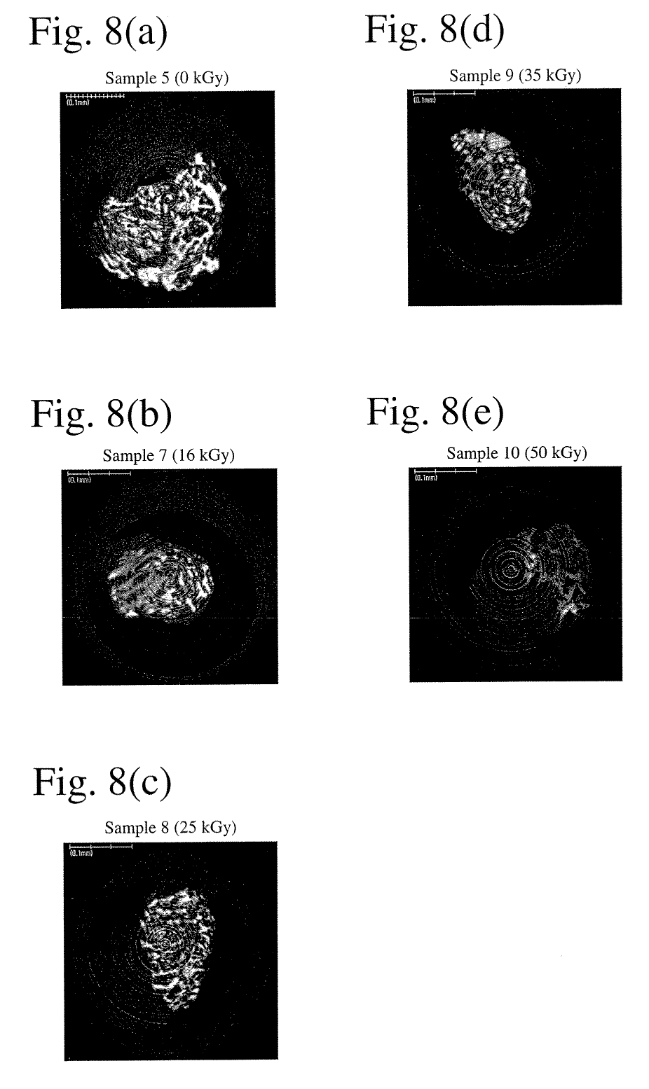

The CT photographs of Samples two weeks after embedded in a muscle layer are shown in FIGS. 8(a)-8(e). In each photograph, a white portion indicates the embedded porous body. Absorption mode was different between Sample 9 (35 kGy) and Sample 10 (50 kGy), whose volume reduction ratios were substantially the same, Sample 9 being absorbed gradually from its periphery [FIG. 8(d)], while Sample 10 was absorbed unevenly [FIG. 8(e)]. Uneven absorption as in Sample 10 fails to regenerate the bone well.

TABLE 2

| | Irradiation Dose | Half-Value Period | Volume Reduction |
|---|---|---|---|
| No.⁽¹⁾ | (kGy) of γ Rays | of Strength (hr.) | Ratio (%) |
| Sample 5 | 0 | 1.79 | 4 |
| Sample 6 | 10 | 1.54 | —⁽²⁾ |
| Sample 7 | 16 | 1.41 | 14 |
| Sample 8 | 25 | 1.21 | 34 |
| Sample 9 | 35 | 0.96 | 69 |
| Sample 10 | 50 | 0.60 | 64 |

Note:
⁽¹⁾Samples 6, 8 and 10 correspond to Samples 1-3 in Example 1, and Samples 6-9 are within the range of the present invention, while Samples 5 and 10 are outside the range of the present invention.
⁽²⁾Not measured.

The above results indicate that the porous body comprising apatite/collagen composite fibers, which has a half-value period of strength of 0.8-1.6 hours, is an optimum material for bone formation. Such porous body can be obtained by the irradiation of γ rays in a dose of 10-42 kGy.

### EFFECT OF THE INVENTION

Because the porous body of the present invention comprising apatite/collagen composite fibers has well-balanced mechanical strength and biocompatibility in the human body, it can be suitably used for artificial bone, cell scaffolds, etc.

The present disclosure relates to subject matter contained in Japanese Patent Application No. 2007-285469 filed on Nov. 1, 2007, which is expressly incorporated herein by reference in its entirety.

What is claimed is:

1. A porous body comprising apatite/collagen composite fibers, which has a half-value period of strength of 0.8-1.6 hours, the half-value period of strength being the time until the strength of the porous body of 10 mm×10 mm×4 mm comprising apatite/collagen composite fibers is reduced to half, after the porous body degassed by pressure reduction to 3 kPa (absolute pressure) for 10 minutes in a phosphate buffer saline is given a 20-% strain at a speed of 10 mm/minute.

2. The porous body comprising apatite/collagen composite fibers according to claim 1, wherein said half-value period of strength is 0.9-1.5 hours.

3. The porous body comprising apatite/collagen composite fibers according to claim 1, wherein the mass ratio of said apatite to said collagen is 9/1-6/4.

4. The porous body comprising apatite/collagen composite fibers according to claim 1, wherein it is irradiated with γ rays in a dose of 10-42 kGy.

5. The porous body comprising apatite/collagen composite fibers according to claim 4, wherein the irradiation dose of γ rays is 16-35 kGy.

6. A method for producing a porous body comprising apatite/collagen composite fibers, which has a half-value period of strength of 0.8-1.6 hours, the half-value period of strength being the time until the strength of the porous body of 10 mm×10 mm×4 mm comprising apatite/collagen composite fibers is reduced to half, after the porous body degassed by pressure reduction to 3 kPa (absolute pressure) for 10 minutes in a phosphate buffer saline is given a 20-% strain at a speed of 10 mm/minute, comprising the steps of obtaining the porous body comprising apatite/collagen composite fibers by freeze-drying, and irradiating the porous body with γ rays in a dose of 10-42 kGy.

7. The method for producing a porous body comprising apatite/collagen composite fibers according to claim 6, wherein a cross-linking treatment is conducted before the irradiation of γ rays.
